(19) United States
(12) Patent Application Publication
(10) Pub. No.: US 2025/0281231 A1
(43) Pub. Date: Sep. 11, 2025

(54) DEVICES AND METHODS FOR ABLATION OF TISSUE

(71) Applicants: Mayo Foundation for Medical Education and Research, Rochester, MN (US); Access Point Technologies EP, Incorporated, Rogers, MN (US)

(72) Inventors: Christopher V. DeSimone, Rochester, MN (US); Samuel J. Asirvatham, Rochester, MN (US); Ammar M. Killu, Rochester, MN (US); David R. Holmes, Jr., Rochester, MN (US); Jason A. Tri, Rochester, MN (US); Derick Sana Berhow, Clearwater, MN (US); Douglas Scott Wahnschaffe, Monticello, MN (US); William Joseph Hart, White Bear Township, MN (US); Steven Wayne Berhow, Clearwater, MN (US)

(21) Appl. No.: 18/859,707

(22) PCT Filed: Apr. 27, 2023
(86) PCT No.: PCT/US2023/020187

§ 371 Date: Oct. 24, 2024

Related U.S. Application Data
(60) Provisional application No. 63/336,059, filed on Apr. 28, 2022.

(51) Int. Cl. A61B 18/14; A61B 18/00

(52) U.S. Cl. CPC A61B 18/1492; A61B 2018/0016; A61B 2018/00184; A61B 2018/00267; A61B 2018/00351; A61B 2018/00375; A61B 2018/00404; A61B 2018/00577; A61B 2018/1475

(57) ABSTRACT

This document provides devices and methods for the treatment of heart conditions, hypertension, and other medical disorders. For example, this document provides devices and methods for treating atrial fibrillation by performing thoracic vein ablation and/or electroporation procedures, including pulmonary vein myocardium ablation. In some embodiments, the ablation and/or electroporation is delivered in differing areas in and/or around the pulmonary vein in coordination with each other for enhanced efficacy.

### CROSS-REFERENCE TO RELATED APPLICATIONS

This application claims the benefit of U.S. Provisional Application Ser. No. 63/336,059, filed Apr. 28, 2022. The disclosure of the prior application is considered part of (and is incorporated by reference in) the disclosure of this application.

### BACKGROUND

### 1. Technical Field

This document relates to devices and methods for the treatment of medical disorders including heart conditions and hypertension. For example, among other things this document relates to devices and methods for treating atrial fibrillation by performing thoracic vein ablation procedures, including pulmonary vein myocardium ablation.

### 2. Background Information

Atrial fibrillation is an irregular and often rapid heart rate that commonly causes poor blood flow to the body. During atrial fibrillation, the heart's two upper chambers (the atria) beat chaotically and irregularly—out of coordination with the two lower chambers (the ventricles) of the heart. Atrial fibrillation symptoms include heart palpitations, shortness of breath, and weakness.

Ablation procedures, including ablation of thoracic veins such as the pulmonary vein, are a treatment for atrial fibrillation. During pulmonary vein ablation, catheters are inserted into the atrium. Energy is delivered from the catheter to the tissue of the pulmonary vein and/or near the ostia of the pulmonary veins in the left atrium. The energy delivered causes scarring of the tissue. The scars block impulses firing from within the pulmonary veins, thereby electrically “disconnecting” them or “isolating” them from the heart. This can provide restoration of normal heart rhythms.

However, undesirable side effects of conventional treatments of atrial fibrillation by pulmonary vein ablation can include pulmonary vein stenosis, neointimal hyperplasia, emboli generation, and electrolysis resulting in bubble formation, to name a few. Pulmonary vein stenosis is the narrowing of the vessels that carry blood from the lungs to the heart. Pulmonary vein stenosis can result in reduced cardiopulmonary efficiency and a decline in quality of life. In some cases, to reduce the effects of stenosis, only a partial circumference of the pulmonary vein is ablated. However, such partial-circumferential ablation procedures are generally less effective for eliminating atrial fibrillation in comparison to ablation of the entire circumference of the pulmonary veins and/or pulmonary vein ostia.

In some cases, ablation procedures can also be used advantageously in the renal arteries to treat hypertension. Ablation of the renal sympathetic nerves using catheter-delivered radiofrequency energy may be an effective intervention for uncontrolled hypertension in some instances. For example, such renal denervation procedures may be beneficial for at least some of the 20 to 30 percent of adults being treated for hypertension that do not achieve adequate blood pressure control with medications.

### SUMMARY

This document describes devices and methods for treating atrial fibrillation, hypertension, and other medical disorders. Atrial fibrillation can be treated in accordance with the devices and methods provided herein by performing a transcatheter ablation procedure, including a pulmonary vein myocardium ablation procedure. In some embodiments, the ablation can be performed in temporal coordination with the delivery of one or more additional treatment modalities such as, but not limited to, tissue stretching, suction, and/or the supply of a pharmacological agent to reduce the occurrence of vein stenosis or neointimal hyperplasia.

In some embodiments, the devices and methods described herein deliver non-thermal energy for ablation using electroporation. The use of electroporation for treating AF is driven by a potentially superior safety and efficacy profile compared to radiofrequency or cryoablation. This non-thermal energy source induces cell death through a mechanism of irreversible electroporation (IRE). The technology described herein delivers pulsed electrical fields (PEF) for short periods (e.g., microseconds to nanoseconds) to cause pores in the cell membrane resulting in disruption of homeostasis and activation of the apoptotic cascade. Unlike, thermal AF treatment modalities, IRE has the potential to induce cell death with a lower risk of collateral damage to the phrenic nerve, coronary arteries, conduction system, or neighboring myocardium. In some embodiments, the PEF is delivered by the various electrodes on the devices in a prescribed and pre-programmed pattern. For example, a first pair of electrodes can be activated to deliver a certain number of PEF, and then another pair can be activated to deliver a certain number of PEF, and so on. Many different patterns of electrode activations are envisioned and within the scope of this disclosure.

In one aspect, this document describes a medical device system for delivering ablation (e.g., RF, microwave, cryoablation) and/or electroporation energy to a tissue of a patient. The system can include an elongate outer catheter shaft defining a first lumen and a longitudinal axis, a self-expanding wire framework comprising a proximal end attached to a distal end portion of the outer catheter shaft and a distal end attached to a nose cone, and an elongate inner catheter slidably disposed in the first lumen and defining a second lumen. A distal end portion of the inner catheter being fixedly attached to the nose cone. The wire framework includes a plurality of wires and a plurality of electrode on the plurality of wires. The plurality of wires each individually extend between the distal end portion of the outer catheter shaft and the nose cone. The plurality of wires are reconfigurable between a low-profile delivery configuration and a radially expanded configuration. In the expanded configuration, each of the wires includes four wire portions serially arranged along a proximal-to-distal direction. The four wire portions include: (i) a first wire portion extending radially outward from the distal end portion of the outer catheter shaft, (ii) a second wire portion extending radially inward, (iii) a third wire portion extending generally parallel to the longitudinal axis, and (iv) a fourth wire portion extending radially inward to the nose cone. Each of the wires includes at least one electrode attached to the second wire portion and at least one electrode attached to the third wire portion.

Such a medical device system for delivering ablation and/or electroporation energy to a tissue of a patient may optionally include one or more of the following features. The system may also include a guidewire slidably disposable in the second lumen and distally extendable through and beyond the nose cone. The system may also include a delivery sheath defining a lumen configured for radial containment of the plurality of wires in the low-profile delivery configuration. The system may also include a hood catheter with an expandable distal end portion that is conical when expanded and configured for contacting and conforming with/against a tissue wall around an ostium with which the wire framework is engageable. The expandable distal end portion may be sized and/or shaped to contain (e.g., radially and longitudinally) a proximal end portion of the plurality of wires when the plurality of wires are in the radially expanded configuration. The hood catheter may be configured to prevent blood flow from passing through a wall of the hood catheter. The hood catheter may be configured to perform filtering by allowing blood flow through a wall of the hood catheter while not allowing embolic material (e.g., char, coagulum, and bubbles) to pass therethrough. The system may also include at least one electrode attached to the outer catheter shaft. The system may also include at least one electrode attached to the nose cone. The plurality of electrodes may include at least two electrodes attached to the third wire portion. In some embodiments, the plurality of wires can be reconfigured between the low-profile delivery configuration and the expanded configuration by longitudinally sliding the inner catheter relative to the outer catheter.

In another aspect, this disclosure is directed to a method for delivering ablation and/or electroporation to one or more tissues of a patient. The method includes advancing any of the medical device systems described herein into the patient and engaging the wire framework with a pulmonary vein of the patient. The first and second wire portions of the wire framework are positioned in a left atrium of the patient and the third and fourth wire portions are in the pulmonary vein. The method further includes energizing at least some of the plurality of electrodes. The energizing provides an energy sufficient for ablation or electroporation of at least some tissue of the pulmonary vein or around the pulmonary vein.

Such a method may optionally include one or more of the following features. The method may also include stretching, by the wire framework, the at least some tissue of the pulmonary vein or around the pulmonary vein, wherein the stretching occurs simultaneously with the energizing. The step of energizing may include using the at least one electrode attached to each second wire portion as a cathode, and using the at least one electrode attached to each third wire portion as an anode. The step of energizing may include using the at least one electrode attached to each second wire portion as an anode, and using the at least one electrode attached to each third wire portion as a cathode. The step of energizing may include delivering RF energy from the at least one electrode attached to each second wire portion, and delivering pulsed DC energy from the at least one electrode attached to each third wire portion as a cathode. The method may also include capturing embolic material simultaneously with the energizing. The step of energizing may provide an energy sufficient for ablation or electroporation of at least some tissue of a wall of the left atrium around an ostium of the pulmonary vein (in addition to the ablation or electroporation of at least some tissue of the pulmonary vein or around the pulmonary vein).

Particular embodiments of the subject matter described in this document can be implemented to realize one or more of the following advantages. Medical conditions such as atrial fibrillation, hypertension, and others can be effectively treated using the devices and methods provided herein. In some embodiments, atrial fibrillation can be treated by pulmonary vein ablation while preventing or reducing stenosis or neointimal hyperplasia of the pulmonary veins during the ablation procedure. In some embodiments, the uptake of the antimitotic pharmacological agent to the tissue receiving the ablation treatment can be promoted using the methods and devices provided herein. In some embodiments, embolic protection is provided by integrating a hood device with the ablation devices provided herein. In such embodiments, the hood device can be used to deliver suction by which the ablation treatment can be enhanced owing to increasing appositional contact forces between the electrodes of the ablation delivery device and the tissue. In some embodiments, a combination of two or more different types of ablation and/or electroporation energy can be delivered using the devices and methods described herein. For example, in some embodiments radiofrequency (RF) energy can be delivered concurrently or sequentially with pulses of direct current (DC) energy. Such delivery of multiple energy types can be leveraged, as described further below, to enhance the overall effects provided by the devices and methods described herein. In some embodiments, various medical conditions can be treated in a minimally invasive fashion using the devices and methods provided herein. Such minimally invasive techniques can reduce recovery times, patient discomfort, and treatment costs.

This document hereby incorporates by reference the disclosures of the following patents and patent applications in their entireties and for all purposes: U.S. patent application Ser. No. 14/892,035 (now U.S. Pat. No. 10,390,879); U.S. patent application Ser. No. 15/771,593 (published as US2018/0360531); and U.S. patent application Ser. No. 16/000,770 (published as US2018/0344393).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### BRIEF DESCRIPTION OF THE DRAWINGS

FIG. 1 is a schematic diagram of a heart undergoing a pulmonary vein ablation procedure using an example catheter-based ablation device in accordance with some embodiments provided herein.

FIG. 2 is an enlarged portion of the schematic diagram of FIG. 1 showing the catheter-based ablation device in greater detail.

FIG. 3 is a cutaway view of FIG. 2 that allows for more visibility of the catheter-based ablation device.

FIG. 4 is a perspective view of a distal end portion of the catheter-based ablation device of FIGS. 1-3.

FIG. 5 is a side view of the distal end portion of the catheter-based ablation device of FIGS. 1-3.

FIG. 6 is a side view of the distal end portion of the catheter-based ablation device of FIGS. 1-3 in a radially contracted configuration.

FIG. 7 is the schematic diagram of FIG. 1 with the addition of a hood catheter in accordance with some embodiments.

FIG. 8 shows another example catheter-based ablation device in accordance with some embodiments.

FIG. 9 shows an enlarged portion of FIG. 8 showing more details of the construction of the catheter-based ablation device.

FIG. 10 shows another example catheter-based ablation device in accordance with some embodiments.

FIG. 11 shows an enlarged portion of FIG. 10 showing more details of the construction of the catheter-based ablation device.

FIG. 12 shows another example catheter-based ablation device in accordance with some embodiments.

FIG. 13 shows a cut away view of the catheter-based ablation device of FIG. 12 being used to treat a pulmonary vein of a heart.

FIGS. 14-16 illustrate an example control handle that can be used in conjunction with the catheter-based ablation devices described herein.

FIG. 17 schematically shows an overall system that can be used in conjunction with the catheter-based ablation devices described herein.

Like reference numbers represent corresponding parts throughout.

### DETAILED DESCRIPTION

This document describes devices and methods for the treatment of heart conditions, hypertension, and other medical disorders. For example, among other things this document describes devices and methods for treating atrial fibrillation by performing transcatheter pulmonary vein myocardium ablation and/or electroporation procedures. Unless specifically stated, the terms “ablation” and “electroporation” are used interchangeably herein.

In some implementations, a pharmacological agent is delivered simultaneously, before, and/or after with the application of the energy. However, in some implementations, no delivery of a pharmacological agent is administered directly by the devices provided herein. Rather, in such implementations ablative energy and/or electroporation is/are delivered without the delivery of a pharmacological agent from the device.

While the devices and methods provided herein are primarily described in the context of the treatment of pulmonary veins and/or the left atrial (“LA”) wall to mitigate atrial fibrillation, many other bodily areas and medical conditions may be treated using the concepts provided. For example, the devices and methods provided herein may also be used to treat other thoracic veins, including the superior vena cava, left superior vena cava or its remnants, the azygos vein, and other venous structures. In another example, the devices and methods provided herein may also be used to treat the renal arteries and veins as part of a renal denervation procedure. In addition, the devices and methods provided herein may also be used to treat bodily areas and medical conditions including but not limited to: pulmonary hypertension, appendage ablation; aortic coarctation; esophageal stenosis; bronchial tree, GI lumen and stenotic valve disorders; great vessel ablation for ventricular arrhythmia—as a handheld device for treating skin conditions including hemangiomas, burns, and wrinkles; the retroglossal region—to stiffen tissue and/or treat sleep apnea; peripheral vessels and coronary arteries—to “cholesterol proof” vessels and/or prevent atherosclerosis; gastric vessels or celiac—to lower ischemic threshold so satiety is felt earlier; coronary vessels—to treat vasospasm; herniorrhaphy or hernia repair, and cerebral vessels—to treat migraine. Still further, the devices provided herein may be used for a preventative treatment for coronary atherosclerosis, especially in the left main, proximal LAD, and proximal circumflex. That is, when used in combination with either a lipolytic agent or a calcilytic agent (e.g., diethyl ether) this technique can be used to treat coronary vascular legions. A further application of the devices and methods provided herein is retrouterine access to the fallopian tubes to treat stenosis, for example resulting from inflammatory disease, to keep the lumen open and the endothelium non-disrupted so that fertility is kept intact.

While the embodiments described herein may be described as providing specific types of ablation, it should be understood that a variety of ablation and/or electroporation techniques and energy sources are envisioned for use alone or in combination with any of the devices and methods described herein. For example, monopolar, bipolar, and/or biphasic ablation and/or electroporation techniques can be used. Ablation energy sources such as radiofrequency (RF), direct current (DC), alternating current (AC) in non-cardiac applications, cryogenics, hot solutions, and the like, and combinations thereof, can be used with the devices provided herein. In some embodiments, non-thermal pulsed field ablation/electroporation (“PFA”) is delivered using the devices and methods described herein. Such PFA can preferentially ablate certain targeted tissues with minimal effects on surrounding, non-targeted tissues. In some embodiments, both DC (e.g., for PFA) and RF electrodes can be advantageously used in combination on the devices provided herein. That is, RF electrodes may be included because they are well suited for delivering ablation energy, while DC electrodes may also be included because they are well suited for electroporation and/or as iontophoretic sources for driving pharmacological agents into tissue. The use of DC and RF electrodes in combination can thereby provide a device that provides the benefits of both types of electrodes.

In some embodiments, the electrodes for delivery of the ablation energy are located on the exterior surfaces of the ablation devices. In other embodiments, one or more central electrodes may be additionally located on a catheter or an inner central shaft of the device. In some embodiments, a combination of types of electrodes are included in a single device, as described further below.

Another embodiment can have spikes or spindles on the device that are arranged to wedge into the surrounding tissue such as myocardial tissue. In some embodiments, such spikes or spindles can be metallic and/or made of the same material as the electrodes and may function as electrodes.

Another embodiment has a natural (expanded) shape that is configured to be placed in the left atrial appendage. An ablation can be performed on a wide ring at the ostium of the appendage to electrically isolate the appendage in a simple, straightforward manner. When the device is still inside the appendage it is used as a marker. Epicardial access can be attained and clip electrodes can be placed on the left atrial appendage, as well as the right atrial appendage. This technique provides a stroke prevention therapy where the appendages will be stimulated, but because they are isolated, even if atrial fibrillation were to occur, the atrium will not fibrillate. This technique may provide the benefit that the muscle of the appendage can still be utilized to contribute to left atrial filling, which in turn may contribute to left ventricular filling, despite the presence of atrial fibrillation.

In some embodiments, the device can have electrodes for recording/mapping and/or for pacing, both proximally and distally, as well as along the catheter device's length (on the catheter shaft at the nose or proximal of the wire basket). This arrangement can advantageously enable the use of algorithms that employ impedance measurements and electrogram-derived signals to preferentially deliver dosages of the ablation and/or electroporation energy.

In some embodiments, the ablation devices described herein include a framework or scaffolding that is attached to or disposed on a distal end portion of a catheter. The framework can be self-expandable (e.g., made from a super-elastic material such as, but not limited to, nitinol with shape memory) and can have ablation electrodes disposed on the framework. In some embodiments, to make the framework, a hypo tube, which is initially tubular, is laser cut, expanded, and shape set into a desired configuration.

In some embodiments, provisions for the delivery of suction/aspiration and/or a liquid pharmacological agent for the prevention or reduction of vessel stenosis and neointimal hyperplasia are contemplated. For instance, the drug paclitaxel is an example of one type of an antimitotic pharmacological agent that can be delivered to the tissue undergoing ablation to prevent or reduce fibrosis and stenosis of the tissue. Paclitaxel can be used beneficially because of its rapid uptake and prolonged retention. In some implementations, paclitaxel can be delivered in 3% saline (or similar hypertonic solution) to enhance further its uptake and retention. While paclitaxel is provided as an example, other pharmacological agents can also be used. In other implementations, a high-energy DC shock (e.g., about 2 to 250 Joules) can be applied to the tissue during and/or after exuding the agent to effectively push the agent into the tissue. In addition, prior to delivery or after delivery biologics (e.g., resiniferatoxin, calcium, purified exosome product [for treatment of stiff LA], etc.) may be delivered to help with ablation or prevention of stenosis, etc.

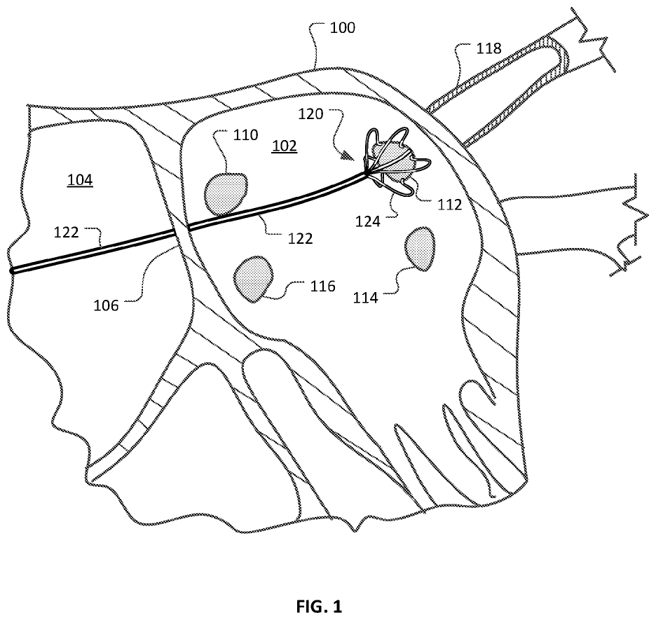

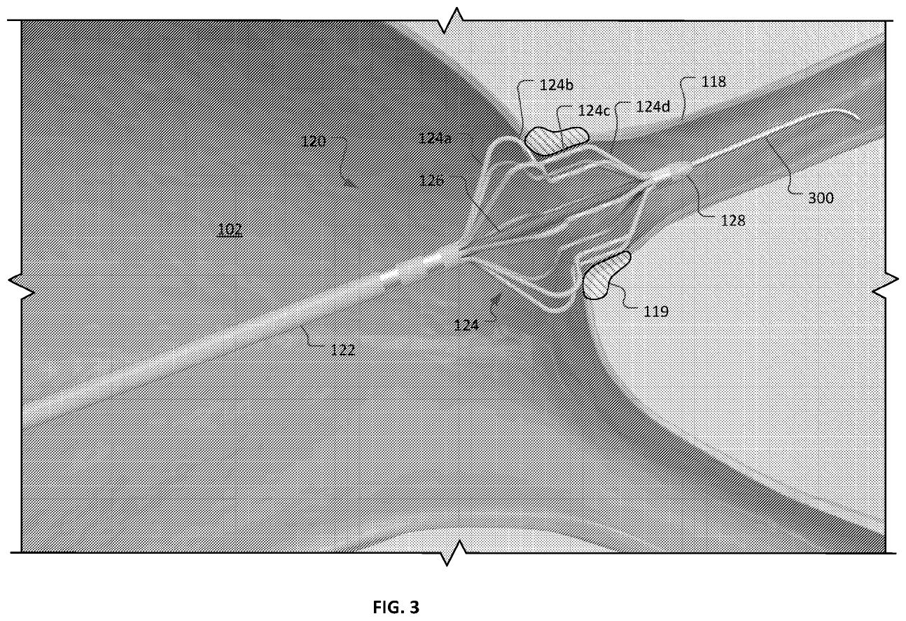

FIG. 1 is a schematic diagram of a pulmonary vein 118 of a heart 100 that is undergoing an ablation procedure using an example catheter device 120 in accordance with some embodiments provided herein. In general, catheter device 120 includes an outer catheter shaft 122 with an example expandable framework 124 attached at a distal end portion of the outer catheter shaft 122. The example expandable framework 124 includes multiple electrodes, as described further below. The multiple electrodes are arranged on the expandable framework 124 to make contact with the atrial wall around the ostium of the pulmonary vein 118 and to make contact with an inner wall of the pulmonary vein 118 (because the expandable framework 124 extends into the pulmonary vein 118 as shown in FIG. 3). The multiple electrodes on the expandable framework 124 and/or the outer catheter shaft 122 are used to deliver ablation and/or electroporation energy to the heart 100 (including the pulmonary vein 118).

During deployment, the distal end of catheter device 120 can be advanced to and positioned in the left atrium 102 of the heart 100 according to standard techniques. For instance, using an example standard technique the catheter device 120 can enter a right atrium 104 of heart 100 through a femoral vein and the inferior vena cava (not shown). The catheter device 120 can then be passed through a puncture or opening in an atrial septum 106 to access the left atrium 102. From the left atrium 102, the catheter device 120 can engage with any of the pulmonary vein ostia 110, 112, 114, or 116 to enter a pulmonary vein (such as the pulmonary vein 118 shown). In some cases, the catheter device 120 can be an over-the-wire device that is delivered over or on a pre-placed guidewire (e.g., as shown in FIG. 3). In some embodiments, the outer catheter shaft 122 can be controllably deflectable in one or more planes to help enable steering of the distal end portion of catheter device 120 during the advancement and placement of the catheter device 120.

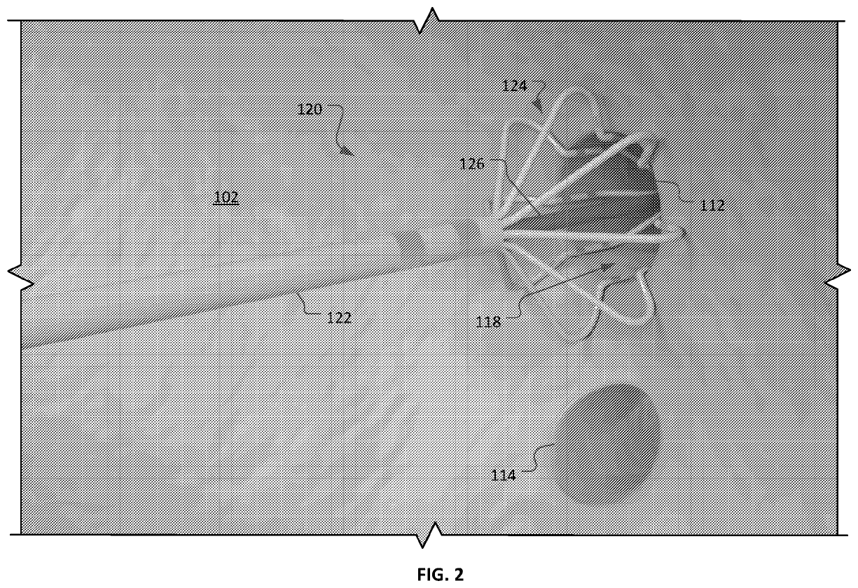

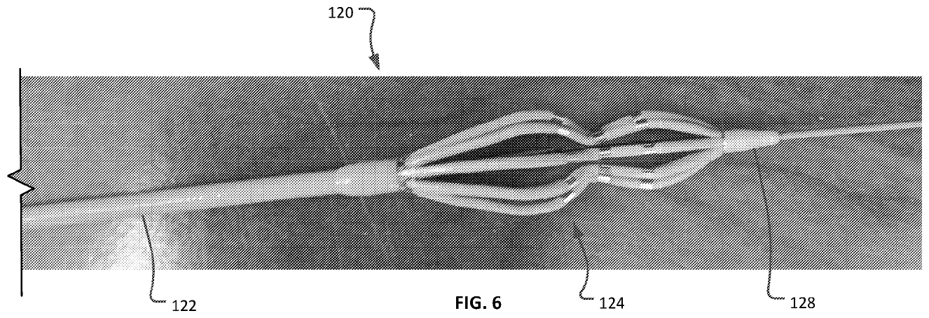

In some cases, a delivery catheter/sheath (not shown) is used to assist in the insertion and placement of catheter device 120. Such a delivery sheath can maintain the expandable framework 124 radially constrained in a low-profile delivery configuration during advancement to the left atrium 102 (e.g., see FIG. 6). Then, in the left atrium 102, the catheter device 120 can be advanced relative to the delivery sheath to cause the expandable framework 124 to emerge from the distal end of the delivery sheath. As the expandable framework 124 emerges from the delivery sheath, the expandable framework 124 will tend to self-expand. Accordingly, the expandable framework 124 can be positioned in engagement with the pulmonary vein 118 and its ostia 112 as depicted (also see FIGS. 2 and 3). In some cases, one or more radiopaque markers can be included on the catheter device 120 and/or the delivery sheath to assist with the radiographical visualization of the advancement and positioning of the catheter device 120 and/or delivery sheath during delivery and deployment.

The proximal end of the catheter device 120 is connected to an ablation energy source and controller (e.g., an RF, DC, and/or AC generator/controller system not shown) which are located external to the patient. Electrodes of the catheter device 120 can be energized with ablation and/or electroporation energy from such a generator/controller system to initiate the modulation of target neural and/or muscle fibers/tissues in and/or around the pulmonary vein 118 and its ostia 112.

An example ablation technique can be generally performed as follows. An electric field can be generated by the external source/controller and transferred through wires within one or more lumens of outer catheter shaft 122 (or the wall of the outer catheter shaft 122, or in the space between the outer catheter shaft 112 and the inner catheter 126) to electrodes disposed on the surface of the expandable framework 124 and/or on the outer surface of the outer catheter shaft 122 or patch(s) placed on the patient skin. The ablation/electroporation energy can be transmitted to the inner wall of pulmonary vein 118 and/or to the wall of the left atrium 102 around the ostia 112. The electric field can modulate the activity along neural fibers within the wall of pulmonary vein 118 and/or the wall of the left atrium 102 around the ostia 112, at least partially denervating the tissue. In some examples, while the electric field for ablation is being applied, a liquid pharmacological agent, suction, or tissue stretching can be concurrently delivered to the tissue. The delivery of a liquid pharmacological agent can provide advantageous results. For example, delivering the agent prior to the ablative energy can provide iontophoresis-like action to drive the agent farther into the tissue. In another example, delivering the ablative energy prior to the pharmacological agent can provide some electroporative disruption of the endothelial cell-to-cell junction, thus promoting the agent delivery. In some implementations, a repetitious cyclic delivery of ablative energy and the pharmacological agent can thereby further enhance uptake of the agent. In some implementations, the pharmacological agent can have an ionic base so as to optimize the ablative energy's ability to get the agent beyond the endothelium of the tissue.

Paclitaxel is an example of one type of antimitotic pharmacological agent that is well suited for this application. This technique of coordinating the delivery of paclitaxel with the ablation process can prevent or reduce the occurrence of fibrosis, stenosis, and neointimal hyperplasia of the tissue undergoing ablation. In such fashion, stenosis of pulmonary vein 118 can be reduced or prevented while full-circumferential ablation of pulmonary vein 118 is performed. Calcium and other types of biologic or non-biologic agents can also be delivered in some embodiments.

Referring to FIGS. 2 and 3, here the shape and construction of the example catheter device 120 can be view in greater detail. Additionally, the engagement of the example expandable framework 124 with the pulmonary vein 118 and its ostia 112 can be visualized more clearly.

The catheter device 120 includes the outer catheter shaft 122. The outer catheter shaft 122 defines a first lumen and a longitudinal axis. The catheter device 120 also includes the expandable framework 124 (also referred to herein as the wire framework 124). The expandable framework 124 has a proximal end attached to a distal end portion of the outer catheter shaft 122. A distal end of the expandable framework 124 is attached to a nose cone 128.

The expandable framework 124 is comprised of a compliant material, or in some cases, a super-elastic material. The super-elastic properties of nitinol make it a good choice for construction the expandable framework 124, however other materials can also be used. The expandable framework 124 can be elastically collapsed to a low-profile configuration for placement within a delivery sheath. Upon emergence from the delivery sheath at the target location within a patient, the expandable framework 124 can self-expand such that the individual wire members of the expandable framework 124 make contact with the tissue of the patient (e.g., the wall tissue of the pulmonary vein 118, renal artery, and/or the left atrium wall tissue near to the ostium 112 of pulmonary vein 118).

The catheter device 120 also includes an elongate inner catheter 126. The inner catheter 126 is slidably disposed in the first lumen of the outer catheter shaft 122. A distal end portion of the inner catheter 126 is fixedly attached to the nose cone 128. The inner catheter 126 defines a second lumen.

In some embodiments, the catheter device 120 can be used in conjunction with a guidewire 300 (FIG. 3). The guidewire 300 can be advanced into position first (so that a distal end portion is positioned in the pulmonary vein 118), and the catheter device 120 can be advanced over the guidewire 300. The second lumen of the inner catheter 126 and the nose cone 128 can slidably contain the guidewire 300. The second lumen of the inner catheter 126 can also be used for various other purposes including, but not limited to, for the delivery of contrast agent, the delivery of biologic or non-biologic agents, and the like.

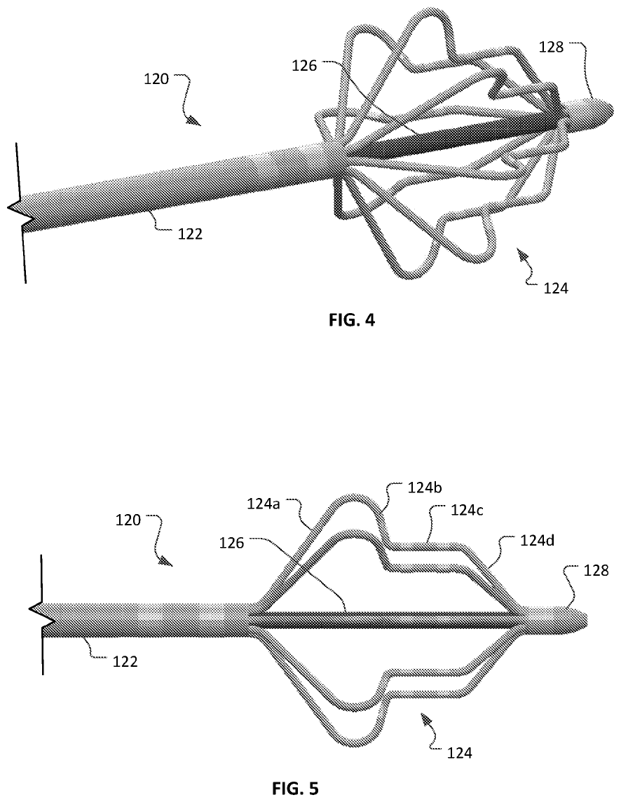

Referring also to FIGS. 4 and 5, the configuration of the example expandable framework 124 and the placement of electrodes on the catheter device 120 will now be described in greater detail.

The example expandable framework 124 is made up of a plurality of wires that individually between the distal end portion of the outer catheter shaft 122 and the nose cone 128. In the depicted embodiment, the expandable framework 124 includes eight individual wires. In some embodiments, two, three, four, five, six, seven, nine, ten, eleven, twelve, or more than twelve individual wires can be included as part of the expandable framework 124.

The example expandable framework 124, as depicted, has a particular shape when in its radially expanded configuration as shown. It should be understood that this is just one example of the type of shape that the expandable framework 124 can be configured to have. For example, in some embodiments the expandable framework 124 can be cylindrical, conical, frustoconical, toroidal, and the like, and any combinations thereof.

In the expanded configuration, each of the wires of the expandable framework 124 includes four wire portions serially arranged along a proximal-to-distal direction. As shown in FIGS. 3 and 5, each wire includes the following four wire portions serially arranged along a proximal-to-distal direction: (i) a first wire portion 124a extending radially outward from the distal end portion of the outer catheter shaft 122, (ii) a second wire portion 124b extending radially inward, (iii) a third wire portion 124c extending generally parallel (e.g., +/−5°, +/−10°, or +/−20°) to the central longitudinal axis of the catheter shaft 122, and (iv) a fourth wire portion 124d extending radially inward to the nose cone 128.

When the expandable framework 124 is engaged with the pulmonary vein 118 and its ostium 112 (e.g., as shown in FIGS. 2 and 3), the first wire portion 124a and the second wire portion 124b are in the left atrium 102 and in contact with the wall of the left atrium 102 around the ostium 112 of the pulmonary vein 118. In contrast, when the expandable framework 124 is engaged with the pulmonary vein 118 and its ostium 112, the third wire portion 124c and the fourth wire portion 124d are in the pulmonary vein 118. This arrangement allows for the delivery of ablation/electroporation energy to a tissue region that extends between the wall of the left atrium 102 around the ostium 112 and the wall of the pulmonary vein 118.

The catheter device 120 includes multiple electrodes that can be used for the delivery of energy for ablation/electroporation, mapping, and/or pacing. In the example use context illustrated in FIGS. 1-3, the electrodes of the catheter device 120 are used for the delivery of various types of energy for ablation and/or electroporation. The electrodes can be used for monopolar, bipolar, and/or biphasic ablation and/or electroporation techniques, or combinations thereof. The electrodes can be activated and/or used in pairs, groups of any quantity, or singularly. Any of the electrodes can be a cathode. Any of the electrodes can be an anode. In some embodiments, the electrodes can switch polarities during a treatment. In some embodiments, only certain ones of the electrodes are activated, thereby providing a desired pattern of ablation (e.g., linear, circular, and the like). In some embodiments, both DC and RF electrodes can be advantageously used in combination on a single catheter device 120. That is, RF electrodes may be included because they are well suited for delivering ablation energy (e.g., for denervating the wall tissue of the left atrium 102 around the ostium 112), while DC electrodes may also be included because they are well-suited to deliver electroporation selectively to pulmonary vein muscle sleeve and/or antrum within or around the pulmonary vein 118, or as iontophoretic sources for driving the pharmacological agents into tissue. The use of DC and RF electrodes in combination can thereby provide a device that provides the benefits of both ablation and electroporation therapies. For example, a low power RF or electroporation can be delivered to the ablation location. This will cause a cellular impedance change to the cells in close contact allowing the full ablative energy delivery to be focused on those cells with a impedance change. This can protect the surrounding structures. This impedance change can be done with cryo, microwave, ultrasound, reversible electroporation, and/or radiofrequency.

In the depicted example, the outer catheter shaft 122 includes two electrodes. However, in some embodiments zero, one, three, four, or more than four electrodes can be included on the outer catheter shaft 122. Additionally, the nose cone 128 includes one electrode in the depicted embodiment. However, in some embodiments zero, two, three, four, or more than four electrodes can be included on the nose cone 128.

In the depicted example, each wire of the expandable framework 124 includes multiple electrodes. In particular, in this example, the second wire portion 124b of each wire includes one electrode and the third wire portion 124c of each wire includes two electrodes. In use, the electrodes of the second wire portions 124b contact the tissue wall of the left atrium 102 around the ostium 122 and the electrodes of the third wire portion 124c extend into the pulmonary vein 118 and contact the inner wall of the pulmonary vein 118. When the electrodes of the second wire portion 124b and the third wire portion 124c (of one wire, of multiple wires, or of all wires of the expandable framework 124) function together as anodes and cathodes, a tissue region around the ostium 112 and extending along the pulmonary vein 118 (as the third wire portion 124c extends) receives the ablation and/or electroporation treatment. This tissue region is depicted by a crosshatched treatment area 119 (shown in FIG. 3), and can extend 360° around the ostium 112 and pulmonary vein 118. However, in some embodiments the treatment area 119 (tissue region treated by the ablation and/or electroporation provided by the catheter device 120) can be less than fully circumferential. For example, depending on which electrodes are utilized, in some embodiments the treatment area 119 can be in a range of 0° to 45°, or 30° to 75°, or 60° to 105°, or 90° to 135°, or 120° to 165°, or 150° to 195°, or 180° to 225°, or 210° to 255°, or 240° to 285°, or 270° to 315°, or 300° to 345° or 0° to 90°, or 90° to 180°, or 180° to 270°, or 270° to 360°, without limitation.

The use of non-RF and non-cryogenic energy (e.g., the use of DC PFA) from the catheter device 120 to deliver ablation/electroporation energy advantageously allows the extension of the treatment area 119 along the pulmonary vein 118 without a risk of causing stenosis of the pulmonary vein 118. In contrast, conventional techniques using RF or cryogenic energy typically cannot be used to treat tissue along the pulmonary vein 118 because there is a concomitant risk of causing stenosis of the pulmonary vein 118.

It should be understood that the depicted arrangement of electrodes on the expandable framework 124 is one of many possible configurations that are contemplated by this disclosure. For example, in some embodiments electrodes can be included on the first wire portions 124a and/or on the fourth wire portions 124d. Moreover, any number of electrodes (e.g., one, two, three, four, five, six, or more than six) can be located on any of the wire portions 124a-d.

FIG. 6 shows the catheter device 120 with its expandable framework 124 in a radially contracted configuration. It should be understood that the expandable framework 124 can be further radially contracted when the expandable framework 124 is positioned within a delivery sheath.

The depicted configuration can be advantageously attained by pushing the inner catheter 126 (not visible) distally relative to the outer catheter shaft 122. Since the nose cone 128 is attached to the distal end of the inner catheter 126, and the distal ends of the wires of the expandable framework 124 are attached to the nose cone 128, the distal movement of the inner catheter 126 relative to the outer catheter shaft 122 will move the nose cone 128 distally from the outer catheter shaft 122. Moreover, the distal movement of the nose cone 128 relative to the outer catheter shaft 122 will longitudinally extend the wires of the expandable framework 124 (contracting the radial outer profile of the expandable framework 124 by moving the wires radially inward toward the inner catheter 126). Inversely, by pulling the inner catheter 126 proximally relative to the outer catheter shaft 122 the expandable framework 124 will be made to expand radially. In fact, the expandable framework 124 can be radially expanded and longitudinally shortened (and flattened) beyond what is shown in FIGS. 4 and 5 by pulling the inner catheter 126 proximally relative to the outer catheter shaft 122. Such a flattened configuration can be used, for example, to deliver ablation/electroporation to the wall of the left atrium 102 without extending into the pulmonary vein 118 (because the shape of the flattened expandable framework 124 becomes essentially toroidal, with a generally planar front face). This treatment technique can be used, for example, to isolate the pulmonary vein 188 or coronary sinus.

This changing/controlling of the shape of the expandable framework 124 by manipulating the inner catheter 126 relative to the outer catheter shaft 122 can be useful during the deployment and use of the catheter device 120. For example, as depicted in FIG. 3, in some cases the pulmonary vein 118 can be reshaped and/or stretched radially larger by expanding the radial profile of the expandable framework 124. Such reshaping/stretching can help to ensure improved/robust contact between the electrodes of the expandable framework 124 and the inner wall of the pulmonary vein 118. This is beneficial because not the ostium 112 and/or the pulmonary vein 118 are not perfectly circular/cylindrical in all patients. Moreover, such tissue stretching can lower the electroporation threshold in some cases. Lowering the electroporation threshold can be advantageous to avoid or mitigate the formation of coagulum, the causation of electrolysis, the causation of unwanted muscle damage, for example.

FIG. 7 shows an example hood catheter 200 engaged over the catheter device 120. The hood catheter 200 includes an elongate catheter shaft 210 and an expandable distal end portion 220 that is conical when expanded and configured for contacting and conforming to the topography of the tissue wall of the left atrium 102 around the ostium 112. The expandable distal end portion 220 is sized to radially cover over and contain the expandable framework 124 when the plurality of wires of the expandable framework 124 are in the radially expanded configuration.

In some embodiments, the expandable distal end portion 220 is configured to prevent blood flow from passing through the expandable distal end portion 220. In such a case, suction can be delivered to the pulmonary vein 118 via the hood catheter 200. Such suction can, at least in some cases, advantageously increase the apposition forces between the electrodes of the expandable framework 124 and the surrounding tissues.

In particular embodiments, the expandable distal end portion 220 comprises a porous material that is configured to allow blood flow through the expandable distal end portion 220 while not allowing embolic material to pass therethrough. That is, in some embodiments the expandable distal end portion 220 acts as a filter device to capture potentially harmful embolic materials (e.g., thrombus, blood clots, plaque, tissue fragments, shards or particles of pharmacological agents, and the like). In some cases, such embolic materials may be generated (or may become embolic) as a result of the use of the catheter device 120.

In some embodiments, the pore size of the porous material of the expandable distal end portion 220 can be selected as desired to provide the desired embolic protection while allowing the transmission of blood flow therethrough. For example, in some embodiments the expandable distal end portion 220 may have a pore size in the range of about 40 μm to about 60 μm, about 50 μm to about 70 μm, about 60 μm to about 80 μm, about 70 μm to about 90 μm, about 80 μm to about 100 μm, about 90 μm to about 110 μm, about 100 μm to about 120 μm, about 110 μm to about 130 μm, about 120 μm to about 140 μm, about 130 μm to about 150 μm, about 140 μm to about 160 μm, or greater than 160 μm.

In some embodiments, the peripheral distal end of the expandable distal end portion 220 can include one or more electrodes that can function in coordination with the electrodes of the catheter device 120.

In some embodiments, the expandable distal end portion 220 is self-expanding to enlarge to a size and shape so as to encapsulate the area around pulmonary vein ostia 112. That is, as the expandable distal end portion 220 is made to emerge from or extend from a delivery sheath, in some embodiments the expandable distal end portion 220 will be biased to reconfigure from a low-profile delivery configuration to an expanded configuration as shown.

In some embodiments, the expanded configuration of the expandable distal end portion 220 is generally conical. However, the conical shape is not required. In some embodiments, the expandable distal end portion 220 may expand to other shapes such as, but not limited to, pyramidal, cylindrical, frustoconical, and the like. The expandable distal end portion 220 is configured to maintain full-wall apposition against the topography defined by the tissue surrounding pulmonary vein ostia 112.

After the desired treatment is provided by the hood catheter 200, the expandable distal end portion 220 can be reconfigured back to the collapsed low-profile configuration for removal from the patient. The expandable distal end portion 220 is configured so that, as the expandable distal end portion 220 is collapsed, any emboli present therein remain securely contained within the collapsed expandable distal end portion 220.

The expandable distal end portion 220 can be constructed of various materials and configurations, and can be constructed using various techniques. In some embodiments, the expandable distal end portion 220 comprises a mesh material. In some such embodiments, the mesh material can comprise a Nitinol material. In some such embodiments, the expandable distal end portion 220 comprises a polyester mesh material, a polyurethane mesh material, or another type of synthetic material.

In some embodiments, the expandable distal end portion 220 can comprises a framework of struts and one or more loops. In some embodiments, the framework can be constructed of Nitinol, or another material. A mesh material can be disposed on the framework. For example, in some embodiments a polyester mesh material can be disposed on a Nitinol framework. In some such embodiments, a compliant nitinol ring on the distal end of the expandable distal end portion 220 can be used to support the filter material and to mold against the topography of the tissue surrounding the pulmonary vein ostia 112.

In some embodiments, expandable distal end portion 220 can be additionally or alternatively configured to substantially occlude blood flow around the region of the pulmonary vein ostia 112. In some such embodiments, the pore size of expandable distal end portion 220 can be selected so that expandable distal end portion 220 will occlude all or substantially all blood flow therethrough. By so controlling the blood flow using expandable distal end portion 220, the therapeutic efficacy of the catheter device 120 can be enhanced in some circumstances. For example, in some circumstances the uptake of liquid pharmacological agents and/or the transfer of ablation energy from the catheter device 120 to the surrounding tissue can be enhanced by controlling the blood flow using expandable distal end portion 220.

In some embodiments, portions of expandable distal end portion 220 can be enhanced to provide radiographic visualization of the position and orientation of the expandable distal end portion 220. For example, some embodiments include a loop of radiopaque material (e.g., titanium, tungsten, barium sulfate, zirconium oxide, and the like) around the mouth of the filter to allow for precise positioning and verification of apposition before proceeding with the intervention. Alternatively, or additionally, in some embodiments one or more radiopaque markers can be included on other portions of the expandable distal end portion 220.

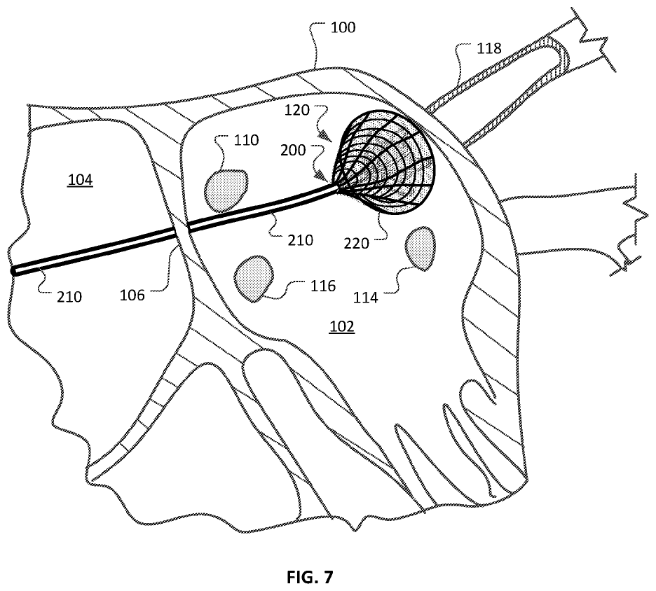

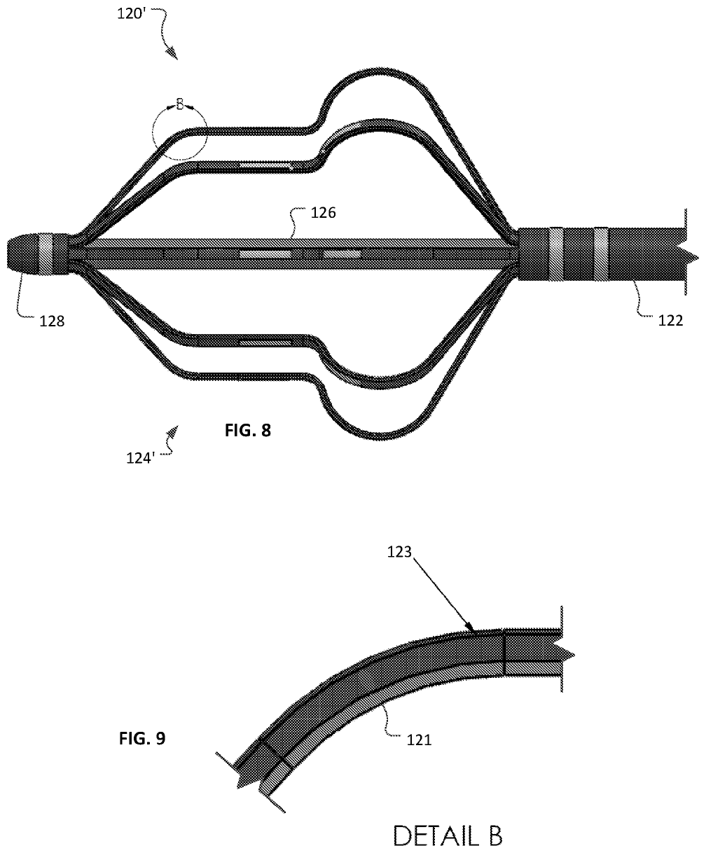

FIGS. 8 and 9 show another example catheter device 120′ in accordance with some embodiments provided herein. In general, catheter device 120′ is a variation of the catheter device 120 described above. However, while the electrodes on the expandable framework 124 of the catheter device 120 are circular metal bands with electrical wires extending proximally to the control handle, the electrodes on the expandable framework 124′ of the catheter device 120′ are pads of flexible printed circuits boards (or simply “flex circuits”). The use of flex circuits for the electrodes of the expandable framework 124′ allows the splines of the expandable framework 124′ to be smaller than the splines of the expandable framework 124. Accordingly, the catheter device 120′ can be radially compressed to a smaller diameter that allows for a smaller delivery profile size.

The catheter device 120′ includes an outer catheter shaft 122 with an example expandable framework 124′ attached at a distal end portion of the outer catheter shaft 122. The example expandable framework 124′ includes multiple electrodes, as described further below. The multiple electrodes are arranged on the expandable framework 124′ to make contact with the atrial wall around the ostium of the pulmonary vein 118 and to make contact with an inner wall of the pulmonary vein 118 (because the expandable framework 124′ extends into the pulmonary vein 118, e.g., as illustrated in FIG. 3). The multiple electrodes on the expandable framework 124′ and/or the outer catheter shaft 122 are used to deliver ablation and/or electroporation energy to the heart 100 (including the pulmonary vein 118).

FIG. 9 shows an enlarged view of a single spline of the expandable framework 124′. In the depicted embodiment, the spline comprises a wire element 121 (e.g., made of shape-memory nitinol, stainless steel, etc.) and a flex circuit 123 attached to the wire element 121. The exposed surfaces of the flex circuit 123 are insulated, except for the electrode pads that are exposed electrically conductive areas. The flex circuit 123 also includes conductors that extend proximally from the electrode pads to the control handle. Accordingly, the use of wires can be eliminated by the use of the flex circuit 123. The exposed electrode pads can be any size and any shape.

In some embodiments, the width of the spline of the expandable framework 124′ (with the flex circuit 123 attached to the wire element 121) is about 0.02 inches or less (0.5 mm or less), or about 0.03 inches or less (0.8 mm or less), without limitation. In some embodiments, the thickness of the spline of the expandable framework 124′ (with the flex circuit 123 attached to the wire element 121) is about 0.004 inches or less (0.1 mm or less), or about 0.006 inches or less (0.15 mm or less), without limitation.

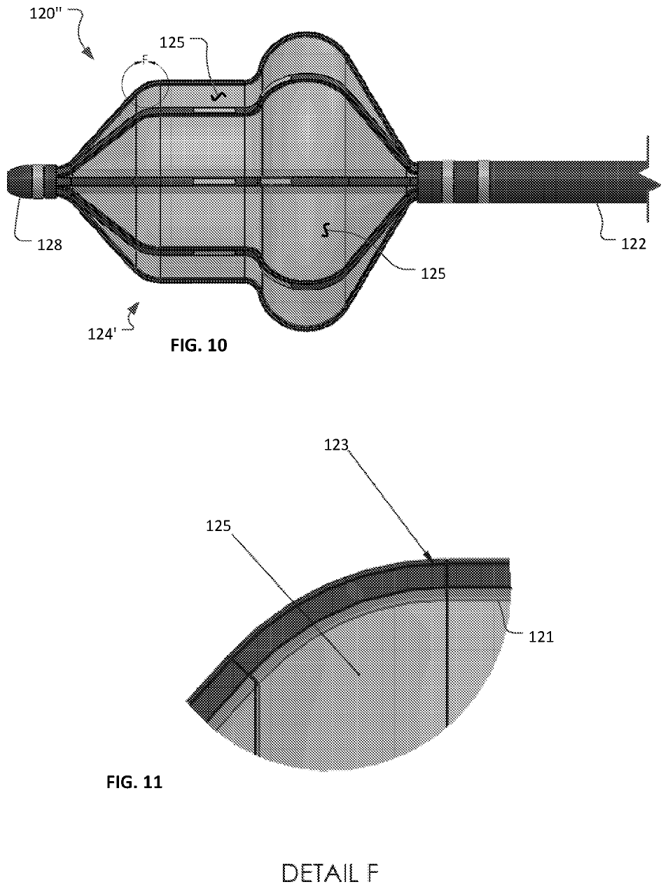

FIGS. 10 and 11 show another example catheter device 120″ in accordance with some embodiments provided herein. In general, catheter device 120″ is a variation of the catheter device 120′ described above. However, the catheter device 120″ includes a balloon 125 located within the expandable framework 124′. The presence of the balloon 125 can help to ensure direct contact by the electrodes of the catheter device 120″ with the tissue that the electrodes are intended to contact. Said another way, the balloon 125 (when inflated as shown) will tend to increase the normal force of the electrodes on the expandable framework 124′ with the surrounding tissue.

The balloon 125 is expandable, in situ, in response to its infilling with a fluid. The fluid can be a liquid (e.g., saline) or a gas (e.g., CO2). The balloon 125 can be made of a compliant material, semi-compliant material, or non-compliant material. The expanded shape of the balloon 125 can correspond to the shape of the expandable framework 124′.

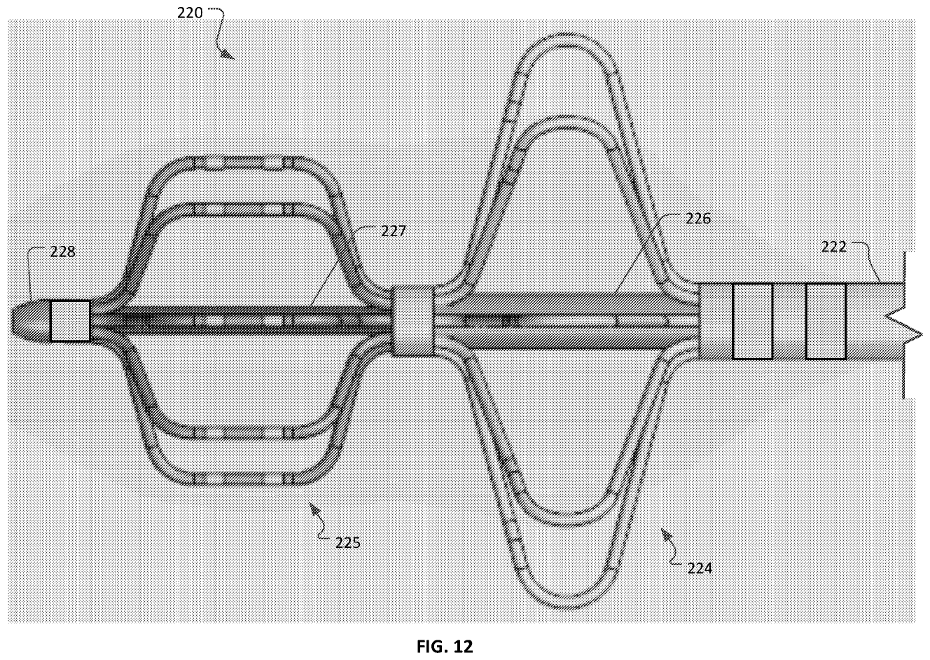

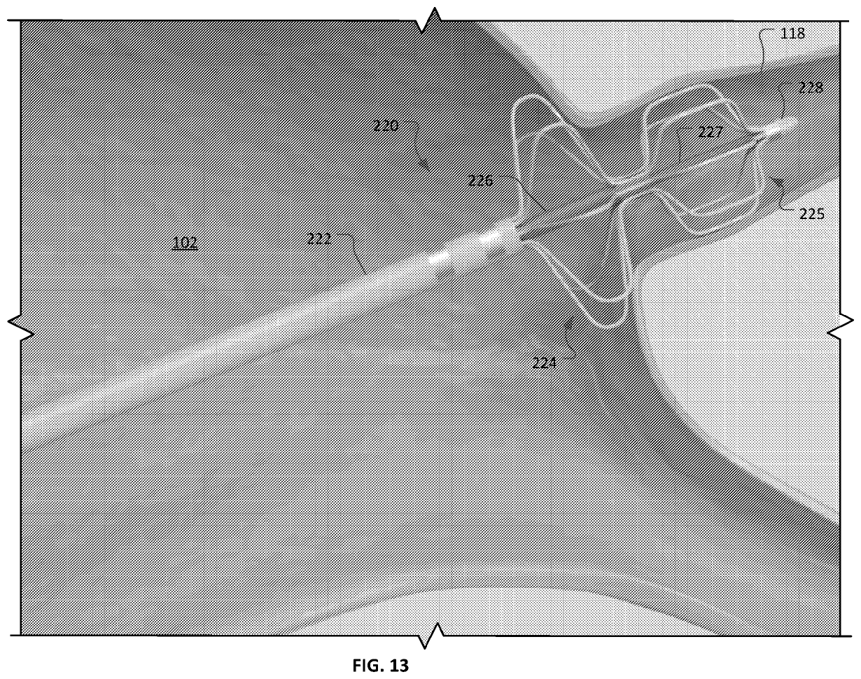

FIGS. 12 and 13 show another example catheter device 220 in accordance with some embodiments provided herein. The catheter device 220 includes an outer catheter shaft 222. A first inner catheter 226 is slidably disposed within the lumen of the outer catheter shaft 222. A second inner catheter 227 is slidably disposed within the lumen of the first inner catheter 226.

The catheter device 220 also includes a first expandable framework 224 and a second expandable framework 225. The proximal end of the first expandable framework 224 is attached to a distal end of the outer catheter shaft 222. A distal end of the first expandable framework 224 is attached to a distal end of the first inner catheter 226. A proximal end of the second expandable framework 225 is attached to a distal end of the first inner catheter 226. A distal end of the second expandable framework 225 (and the nose cone 228) is attached to a distal end of the second inner catheter 227. Accordingly, the expansion and contraction of the first expandable framework 224 and the second expandable framework 225 can be individually controlled by the relative positioning of the first inner catheter 226 and the second inner catheter 227 in relation to each other, and in relation to the outer catheter shaft 222. For example, the first expandable framework 224 can be expanded while the second expandable framework 225 remains contracted, or vice versa.

The expandable frameworks 224 and 225 each include multiple electrodes. The multiple electrodes are arranged on the first expandable framework 224 to make contact with the atrial wall around the ostium of the pulmonary vein 118. The multiple electrodes are arranged on the second expandable framework 225 to make contact with an inner wall of the pulmonary vein 118 (because the second expandable framework 225 extends into the pulmonary vein 118, e.g., as illustrated in FIG. 13). The multiple electrodes on the expandable frameworks 224 and 225 and/or the outer catheter shaft 222 and/or the nose cone 228 are used to deliver ablation and/or electroporation energy to the heart 100 (including the pulmonary vein 118).

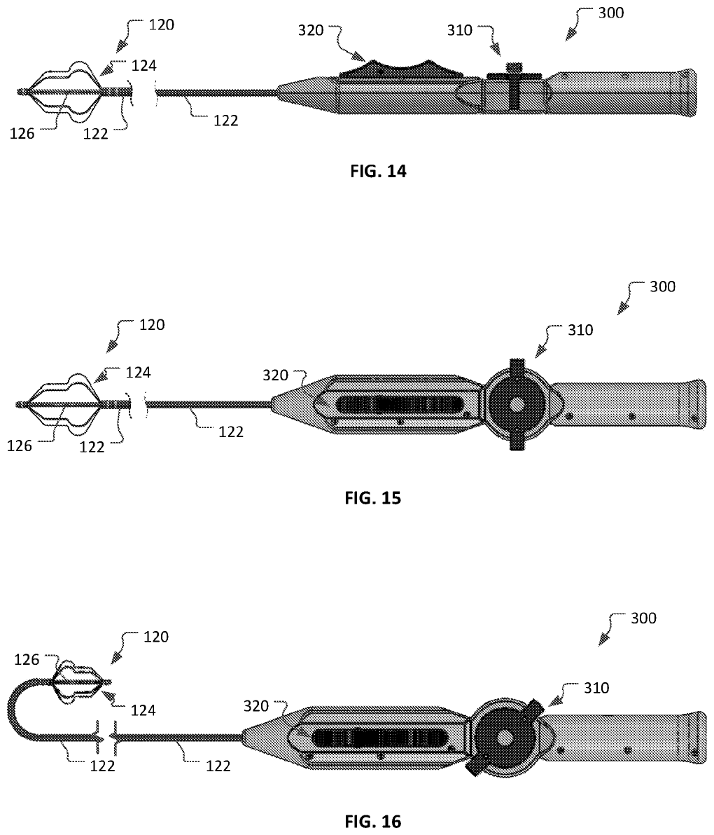

FIGS. 14-16 illustrate an example control handle 300 that can be used to deploy and control any of the catheter devices described herein. In these figures, the catheter device 120 is depicted as a non-limiting example.

The control handle 300 includes a rotary mechanism 310 that can be actuated to deflect the outer catheter shaft 122. To facilitate this, in some embodiments, pull wires are attached to the rotary mechanism 310, and extend through lumens in the wall of the outer catheter shaft 122, terminating at a distal end portion of the outer catheter shaft 122. Accordingly, actuation of the rotary mechanism 310 will tension one pull wire and relax another pull wire to cause the deflection of the outer catheter shaft 122 as depicted in FIG. 16.

The control handle 300 also includes one or more slider mechanisms 320 that can be actuated to expand and retract the expandable framework 124. The one or more slider mechanisms 320 can be attached to the inner catheter 126 and can extend and retract the inner catheter 126 relative to the outer catheter shaft 122 (thereby expanding and contracting the expandable framework 124).

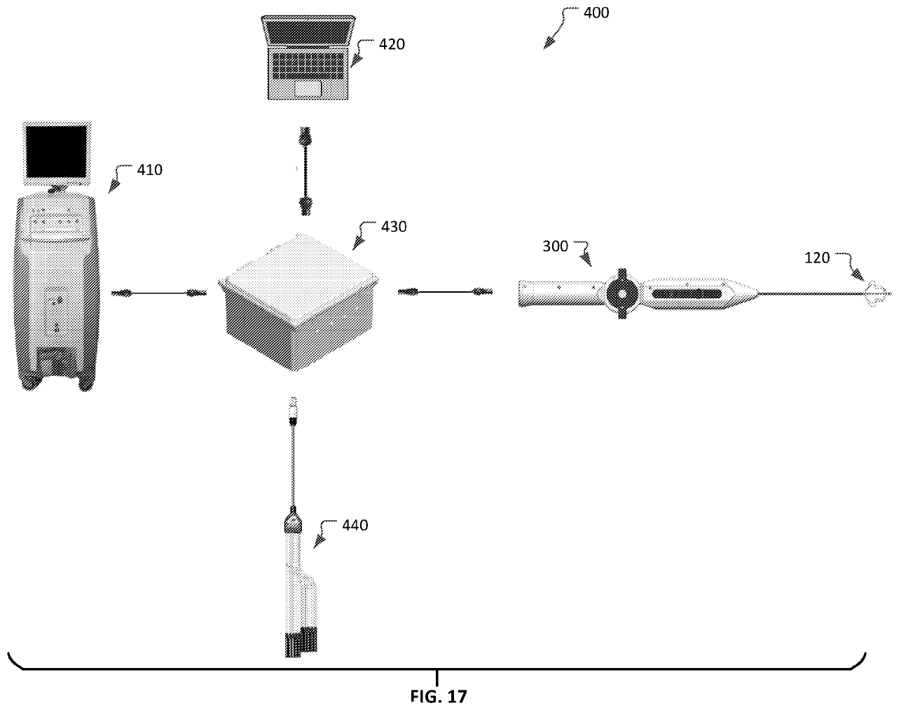

FIG. 17 schematically shows an example system 400 that can be used to operate the catheter devices described herein. The system 400 includes a DC energy system 410, a control system 420 (e.g., computerized system capable of selecting any electrode firing configuration), a multiplexor 430, a connection to a recording system (e.g., for capturing electrograms, mapping, and/or pacing), and the example catheter device 120 with its handle 300.

The multiplexor 430 is used to select any of the electrodes on the catheter that are not pre-programed to delivery. This allows the user to select any electrode configuration and firing pattern. The electrode activation patterns/configurations are controlled by one or more user-selectable software programs stored in the control system 420. Control system 420 and multiplexor 430 can also be used in conjunction with the DC energy system 410 to turn on and off the electrode sensing/mapping ability when delivering the DC energy for electroporation. This will protect the recording system from high voltage energy shocks. This is done with a digital switching system used on the circuit boards within the multiplexor 430. The switching program is housed within the control system 420 and can be programmed to turn and off based on the current firing pattern of the DC energy system 410 during DC energy delivery.

### ADDITIONAL OPTIONAL FEATURES AND EMBODIMENTS

In some embodiments, one or more piezoelectric crystals is/are attached on the distal end of the frame, middle of the expanded frame, and/or the proximal area of the shaft. A piezoelectric crystal may also be placed on the distal end of a wire that may subsequently be placed in an adjacent structure or in the LA atrium.

Any of the electrodes described herein can be used as sensing electrodes, pacing electrodes, and anodes and/or cathodes for delivery of PEF and PFA. The DC pulses delivered by the electrodes of the catheter devices described herein can be monophasic and/or biphasic. Any pulse duration can be used. Any number of pulses can be delivered in a train of pulses. Any of the electrodes can be fired in any desired pattern. In some embodiments, pulses are delivered in synchronization/coordination with the patient's ECG.

The catheter devices described herein can be used in various other areas in addition to the pulmonary vein. Such other areas can include, but are not limited to, the coronary sinus, left atrial appendage, right atrial appendage, left ventricular outflow tract, right ventricular outflow tract, aorta, superior vena cava, inferior vena cava, and so on.

It should also be understood, that the features and usage techniques described herein in relation to the various ablation devices can be combined with the features of other ablation device embodiments and usage techniques described herein. Accordingly, based on such combinations and sub-combinations, an extensive number of ablation device embodiments and usage techniques are envisioned and provided herein.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A medical device system for ablating a tissue of a patient, the system comprising:
an elongate outer catheter shaft defining a first lumen and a longitudinal axis;
a self-expanding wire framework comprising a proximal end attached to a distal end portion of the outer catheter shaft and a distal end attached to a nose cone; and
an elongate inner catheter slidably disposed in the first lumen and defining a second lumen, a distal end portion of the inner catheter being fixedly attached to the nose cone,
wherein the wire framework further comprises:
a plurality of wires that each individually extend between the distal end portion of the outer catheter shaft and the nose cone, the plurality of wires being reconfigurable between a low-profile delivery configuration and a radially expanded configuration, wherein, in the expanded configuration, each of the wires includes four wire portions serially arranged along a proximal-to-distal direction and comprising: (i) a first wire portion extending radially outward from the distal end portion of the outer catheter shaft, (ii) a second wire portion extending radially inward, (iii) a third wire portion extending generally parallel to the longitudinal axis, and (iv) a fourth wire portion extending radially inward to the nose cone; and
a plurality of electrodes on the plurality of wires, wherein each of the wires includes at least one electrode attached to the second wire portion and at least one electrode attached to the third wire portion.

2. The system of claim 1, further comprising a guidewire slidably disposable in the second lumen and distally extendable through and beyond the nose cone.

3. The system of claim 1, further comprising a delivery sheath defining a lumen configured for radial containment of the plurality of wires in the low-profile delivery configuration.

4. The system of claim 1, further comprising a hood catheter with an expandable distal end portion that is conical when expanded and configured for contacting and conforming with a tissue wall around an ostium with which the wire framework is engageable, wherein the expandable distal end portion is sized to radially contain the plurality of wires when the plurality of wires are in the radially expanded configuration.

5. The system of claim 4, wherein the hood catheter is configured to prevent blood flow from passing through a wall of the hood catheter.

6. The system of claim 4, wherein the hood catheter is configured to allow blood flow through a wall of the hood catheter while not allowing embolic material to pass therethrough.

7. The system of claim 1, further comprising at least one electrode attached to the outer catheter shaft.

8. The system of claim 1, further comprising at least one electrode attached to the nose cone.

9. The system of claim 1, wherein the plurality of electrodes includes at least two electrodes attached to the third wire portion.

10. The system of claim 1, wherein the plurality of wires can be reconfigured between the low-profile delivery configuration and the expanded configuration by longitudinally sliding the inner catheter relative to the outer catheter.

11. The system of claim 1, further comprising a balloon positioned within the wire framework.

12. The system of claim 11, wherein the balloon, in its inflated configuration, is shaped to conform to a shape of the wire framework.

13. A medical device system for ablating a tissue of a patient, the system comprising:
an elongate outer catheter shaft defining a first lumen and a longitudinal axis;
a first inner catheter slidably disposed in the first lumen and defining a second lumen;
a second inner catheter shaft slidably disposed in the second lumen;
a first self-expanding wire framework comprising a proximal end attached to a distal end portion of the outer catheter shaft and a distal end attached to a distal end of the first inner catheter; and
a second self-expanding wire framework comprising a proximal end attached to the distal end of the first inner catheter and a distal end attached to a distal end of the second inner catheter,
wherein the first self-expanding wire framework further comprises:
a plurality of first wires that are reconfigurable between a low-profile delivery configuration and a radially expanded configuration; and
a plurality of first electrodes on the plurality of first wires,
wherein the second self-expanding wire framework further comprises:
a plurality of second wires that are reconfigurable between a low-profile delivery configuration and a radially expanded configuration; and
a plurality of second electrodes on the plurality of second wires.

14. The medical device system of claim 13, wherein the plurality of first electrodes and/or the plurality of second electrodes comprises flexible printed circuit boards.

15. A method for ablating a tissue of a patient, the method comprising:
advancing the system of claim 1 into the patient to engage the wire framework with a pulmonary vein of the patient, wherein the first and second wire portions are in a left atrium of the patient and the third and fourth wire portions are in the pulmonary vein; and
energizing at least some of the plurality of electrodes, wherein the energizing provides an energy sufficient for ablation or electroporation of at least some tissue of the pulmonary vein or around the pulmonary vein.

16. The method of claim 15, further comprising stretching, by the wire framework, the at least some tissue of the pulmonary vein or around the pulmonary vein, wherein the stretching occurs simultaneously with the energizing.

17. The method of claim 15, wherein the energizing comprises:
using the at least one electrode attached to each second wire portion as a cathode; and
using the at least one electrode attached to each third wire portion as an anode.

18. The method of claim 15, wherein the energizing comprises:
using the at least one electrode attached to each second wire portion as an anode; and
using the at least one electrode attached to each third wire portion as a cathode.

19. The method of claim 15, wherein the energizing comprises:
delivering RF energy from the at least one electrode attached to each second wire portion; and
delivering pulsed DC energy from the at least one electrode attached to each third wire portion as a cathode.

20. The method of claim 15, further comprising capturing embolic material simultaneously with the energizing.

21. (canceled)
